(19) United States
(12) Patent Application Publication
(10) Pub. No.: US 2015/0297639 A1
(43) Pub. Date: Oct. 22, 2015

(54) TREATING CANCER

(71) Applicant: Rhode Island Hospital, A Lifespan-Partner, Providence, RI (US)

(72) Inventors: Peter J. Quesenberry, Providence, RI (US); Devasis Chatterjee, Mansfield, MA (US); David R. Mills, Warwick, RI (US); Jason M. Aliotta, Providence, RI (US)

(21) Appl. No.: 14/378,836

(22) PCT Filed: Apr. 18, 2013
(86) PCT No.: PCT/US13/37226

§ 371 Date: Aug. 14, 2014

Related U.S. Application Data
(60) Provisional application No. 61/635,077, filed on Apr. 18, 2012.

(51) Int. Cl. A61K 35/12; A61K 35/42; A61K 35/30; A61K 35/22; A61K 35/407; A61K 35/52; A61K 35/38

(52) U.S. Cl. CPC A61K 35/12; A61K 35/52; A61K 35/42; A61K 35/38; A61K 35/22; A61K 35/407; A61K 35/30

(57) ABSTRACT

The specification provides compositions and methods for treating cancer with non cancerous cell extracellular vesicles. Extracellular vesicles act as a surrogate microenvironment to reverse the drug-resistance or cancerous phenotype of cancer cells towards a non cancerous phenotype.

### CROSS REFERENCE TO RELATED APPLICATIONS

This application claims the benefit of U.S. Application No. 61/635,077, filed on Apr. 18, 2012, the entire content of which are hereby incorporated by reference in its entirety.

### STATEMENT AS TO FEDERALLY SPONSORED RESEARCH

This invention was made with U.S. Government support under Grant Nos. P20GM103421 awarded by the National Institute of General Medical Sciences of the National Institutes of Health, P20 RR 017695 awarded by the National Center for Research Resources, and 8P20GM103421-09 awarded by the National Institute of General Medical Sciences of the National Institutes of Health COBRE Center for Cancer Research Development grant. The Government has certain rights in the invention.

### TECHNICAL FIELD

The claimed compositions and methods relate to treating cancer with non-cancerous cell extracellular vesicles.

### BACKGROUND

In 2008, approximately 12.7 million cancers were diagnosed (excluding non-melanoma skin cancers and other non-invasive cancers) and 7.6 million people died of cancer worldwide (Jemal et al., CA Cancer J Clin 61:69-90, 2011). Cancers as a group account for approximately 13% of all deaths each year with the most common being: lung cancer (1.4 million deaths), stomach cancer (740,000 deaths), liver cancer (700,000 deaths), colorectal cancer (610,000 deaths), breast cancer (460,000 deaths), and prostate cancer (300,000 deaths) (World Health Organization 2012 Statistics). This makes invasive cancer the leading cause of death in the developed world and the second leading cause of death in the developing world.

The dominant therapeutic approaches that are currently employed to treat cancer include surgical removal of primary tumors, tumor irradiation, and parenteral application of anti-mitotic cytotoxic agents. The continued dominance of these long established therapies is mirrored by a lack of improvement in survival rates for most cancers. In addition to limited clinical success, devastating side effects accompany these classic therapies. Both radiation- and cytotoxic-based therapies result in the destruction of rapidly dividing hematopoietic and intestinal epithelial cells leading to compromised immune function, anemia, and impaired nutrient absorption. Surgical intervention often results in a release of tumor cells into the circulation or lymph systems from which metastatic tumors can subsequently be established. Therefore, effective methods and compositions for treating cancer are desirable.

Recent attention has been focused on the task of identifying soluble factors secreted by tumor cells and characterizing their paracrine activities (Bavik et al., Cancer Res 66:794-802, 2006; Chang et al., Proc Natl Acad Sci USA 97:4291-4296, 2000; Currid et al., Proteomics 6:3739-3753, 2006; Krtolica et al., Proc Natl Acad Sci USA 98:12072-12077, 2001; Shay and Roninson, Oncogene 23:2919-2933, 2004). In addition to soluble paracrine factors, many tumor cells also release extracellular vesicles (ECVs), also known as microvesicles (MV5), or exosomes. These vesicles are distinguished by their size (approximately 30 to 1000 nm) and morphology and are secreted by a variety of cell types under physiological and pathological conditions; specifically, they are secreted when a multivesicular endosome fuses with the plasma membrane (Keller et al., Immunol Lett 107:102-108, 2006; Abusamra et al., Blood Cells Mol Dis 35:169-173, 2005). According to previous studies, ECVs can contain bioactive molecules, nucleic acids, proteins, and/or lipids common to the originating cell (Muralidharan-Chari et al., J Cell Sci 123:1603-1611, 2010). Interestingly, the abundance of ECVs released generally correlates positively with advanced grade and stage of cancer progression (Taylor and Gercel-Taylor, Br J Cancer 92:305-311, 2005). Activated cells of various types are known to produce and shed membrane ECVs into their surroundings. However, the mechanism triggering ECV generation by cancer cells is unknown. There is mounting evidence that vesicle trafficking is a highly important process in tumorigenesis. Further evaluation of vesicle trafficking may reveal a number of targets and strategies that may be important for cancer therapeutics. Importantly, ECVs have been shown to affect diverse biologic processes of neighboring cells by altering, for example, cell signaling, cytokine production, angiogenesis, and regulation of immune cell responses (Ratajczak et al., Leukemia 20:1487-95, 2006).

### SUMMARY

The present invention is based, in part, on the discovery that ECVs from non-cancerous cells can be used to transform cancerous cells to reverse drug resistance and sensitize cancerous cells to chemotherapy. In addition, ECVs isolated from malignant biopsied tumor specimens can transform non-malignant cells. The present invention provides, for example, methods of treating cancer using non-cancerous cell ECVs.

Accordingly, the present invention features methods of treating cancer, preventing or inhibiting cancer, or reducing a risk of cancer, e.g., naturally arising cancer, in a subject. The methods include administering to and/or prescribing for a subject selected, e.g., identified or diagnosed, as having, e.g., suffering from, or at risk for developing cancer a therapeutically effective amount of non-cancerous cell extracellular vesicles. The methods can include monitoring the progress of the treatment (e.g., monitoring tumor size or metastasis following and/or during treatment).

In some embodiments, the non-cancerous cell extracellular vesicles are administered by exposing a cancerous tissue to the non-cancerous cell extracellular vesicles. In one embodiment, the non-cancerous cell extracellular vesicles are administered by injecting or infusing a cancerous tissue with the non-cancerous cell extracellular vesicles. In one embodiment, the method comprises isolating non-cancerous cell extracellular vesicles from the subject to be treated or a different subject.

In one embodiment of the present invention, the cancer is prostate cancer, breast cancer, lung cancer, colon cancer, kidney cancer, liver cancer, or brain cancer.

In one embodiment, the non-cancerous cell extracellular vesicles are from non-cancerous cells of the same type as the cancerous cells to be treated in the subject, e.g., wherein the non-cancerous cell extracellular vesicles are extracellular vesicles of non-cancerous prostate, breast, lung, colon, kidney, liver, or brain cells. In one embodiment, the non-cancerous cell extracellular vesicles are from the same subject to be treated. In one embodiment, the non-cancerous cell extracellular vesicles are from a different subject than the subject to be treated.

In one embodiment, the cancer is prostate cancer and the non-cancerous cell extracellular vesicles are prostate cell extracellular vesicles. In one embodiment, the cancer is breast cancer and the non-cancerous cell extracellular vesicles are breast cell extracellular vesicles. In one embodiment, the cancer is lung cancer and the non-cancerous cell extracellular vesicles are lung cell extracellular vesicles. In one embodiment, the cancer is colon cancer and the non-cancerous cell extracellular vesicles are colon cell extracellular vesicles. In one embodiment, the cancer is kidney cancer and the non-cancerous cell extracellular vesicles are kidney cell extracellular vesicles. In one embodiment, the cancer is liver cancer and the non-cancerous cell extracellular vesicles are liver cell extracellular vesicles. In one embodiment, the cancer is brain cancer and the non-cancerous cell extracellular vesicles are brain cell extracellular vesicles.

The methods can be used alone or in combination with other methods for treating cancer in subjects. Accordingly, in one embodiment, the methods described herein can include treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

The subject is an animal, human or non-human, and rodent or non-rodent. For example, the patient can be any mammal, e.g., a human, other primate, pig, rodent such as mouse or rat, rabbit, guinea pig, hamster, cow, horse, cat, dog, sheep or goat, or a non-mammal such as a bird.

Also included in the present invention is a pharmaceutical composition comprising a substantially pure preparation of non-cancerous cell extracellular vesicles and a pharmaceutically acceptable carrier. In one embodiment, the composition comprises extracellular vesicles of non-cancerous prostate, breast, lung, colon, kidney, liver, and/or brain cells. In some embodiments, the composition comprises a nanoparticle.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer, e.g., prostate cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or subject being treated. As is evident to one skilled in the art, a sufficient or significant delay can, in effect, encompass prevention, in that the subject does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. An effective amount can be administered in one or more administrations. In the case of cancer, the effective amount of the drug or composition may: (i) reduce the number of cancer cells; (ii) reduce tumor size; (iii) inhibit, retard, slow to some extent, and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with cancer.

The term “subject” refers to an animal, human or non-human, and rodent or non-rodent. For example, the subject can be any mammal, e.g., a human, other primate, pig, rodent such as mouse or rat, rabbit, guinea pig, hamster, cow, horse, cat, dog, sheep or goat, or a non-mammal such as a bird. The cancer can be the result of any of a number of factors, e.g., carcinogens; infections, e.g., viral infections; radiation; and/or heredity, or can be of indeterminate origin. The pharmaceutical composition can be in any form, e.g., gaseous or liquid form.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D are a series of photomicrographs and bar graphs showing ECV-mediated reversal of apoptosis resistance and sensitivity. A, B. ECVs were isolated from DU145 S and DU145 R prostate cells. The ECVs were resuspended in PBS. DU145 S cells were co-cultured with DU145 R ECVs and DU145 R cells were co-cultured with DU145 S ECVs. Non-ECV and ECV co-cultured cells were treated with 100 nM of the anticancer agent camptothecin (CPT) for 24 hours and examined for PARP cleavage and actin via Western blot analysis to measure apoptosis. C, D. The same experiment from A and B was repeated and the samples were examined for apoptosis via propidium iodide staining using a flow cytometer. The data is the mean+/−s.d. of two independent experiments performed in triplicate.

FIG. 2 is a bar graph depicting ECV-mediated reversal of soft agar growth. ECVs were isolated from PrECs and DU145 cells. PrEC cells were co-cultured with DU145 ECVs and DU145 cells were co-cultured with PrEC ECVs for three days. Cells were harvested and utilized for soft agar cloning. Soft agar cloning was examined using 0.7% agarose in PBS and mixed with 1× media with 10% FBS. The top layer consisted of 0.35% agarose in PBS, 1× media with 10% FBS, and 1×10⁵ cells per dish. Dishes were incubated at 37° C. and 5% CO₂. After two weeks, cell colonies were counted. Five fields/dish using the 40× objective were counted and there were six dishes/condition.

FIG. 3 is a photomicrograph of a Western blot analysis showing that DU145 cells co-cultured with self-ECVs have enhanced expression of STAT3, while co-culture with PrEC ECVs leads to increased expression of SOCS2.

FIG. 4 is a panel of a bar graph and three photomicrographs showing enhancement of soft agar growth via prostate patient ECVs. ECVs were co-cultured with PrECs for 10 days after which soft agar growth was determined. Six fields/dish were counted and the data represents the mean+/−s.d. of two independent experiments performed in triplicate. A paired t-test was performed to analyze the increase in soft agar colony formation of PrEC cells when co-cultured with ECVs from patient 18, * p<0.005, and patient 19 **p<0.001 when compared to untreated PrEC cells. The photomicrographs are representative of an area of a field that was counted.

FIGS. 5A-D is a panel of a photomicrograph and three Venn diagrams showing a transfer of proteins from patient ECVs. ECVs were co-cultured with PrECs for seven days. A portion of the sample was used for mass spectrometry analysis while another portion was used for Western blot analysis.

FIGS. 6A-C is a panel of a Venn diagram and two bar graphs showing common proteins between patients 13, 14, and 16 and associated functions and canonical pathways. FIG. 6A shows the common and unique proteins between patients 13, 14, and 16. The bar graphs show the significance (−log(p-value)) of specific functions (FIG. 6B) and canonical pathways (FIG. 6C) in each patient. The threshold cutoff of significance is p<0.05 (or −log=1.3).

FIGS. 7A and B are two bar graphs showing common proteins between patients 13, 14, 16, 18, and 19 and associated functions and canonical pathways. IPA analysis of 71 common proteins between patients 13, 14, 16, 18, and 19. The bar graphs show the significance (−log(p-value)) of specific functions (FIG. 7A) and canonical pathways (FIG. 7B) in each patient. The threshold cutoff of significance is p<0.05 (or −log=1.3).

FIG. 8 is a photomicrograph showing MCF7 and MDR breast cancer cells treated with the indicated doses of doxorubicin (Dox) for 24 hours. Western blot analysis was performed to measure the cleavage of PARP (an indicator of apoptosis induction).

FIG. 9 is a photomicrograph of a Western blot analysis for PARP cleavage and actin in protein lysates isolated from MDR (resistant) cells that were co-cultured with MCF7 (sensitive) ECVs for three days then treated with the indicated concentrations of Dox.

FIG. 10 is a bar graph showing the amount of apoptosis in MCF-7 cells that were treated with 10 nM Dox for 24 hours in the presence or absence of MDR ECVs. Cells were harvested and apoptosis measured by flow cytometry. A paired t-test was performed * comparing Dox-treated MCF7 cells and MCF7 cells co-cultured with MDR ECVs. * A paired t-test (p<0.0005) was performed comparing the reduction in soft agar colony formation between Dox treated and MCF7+MDR ECVs+Dox.

FIG. 11 is a bar graph depicting the average number of colonies formed from HBL (non-malignant) and MCF7 (malignant) cells. Co-culture and anchorage-independent growth was performed as described in FIG. 2. A paired test was performed comparing colony formation: * (p<0.000025) between HBL (non-malignant) cells and HBL cells co-cultured with MCF7 (malignant) ECVs and ** (p<0.00000012) demonstrating the reduction in soft agar colony formation between MCF7 cells and MCF7 cells co-cultured with HBL ECVs.

FIG. 12 is a photomicrograph of a Western blot analysis on selected proteins to demonstrate ECV-mediated protein transfer. HBL and MDR breast cells were grown in SILAC medium for five cell doublings. Cells were harvested and lysates combined for mass spectroscopy analysis to determine changes in protein expression.

### DETAILED DESCRIPTION

The present disclosure provides methods to reverse different cellular phenotypes, including cancer, via exposure of cancer cells to ECVs from non-cancerous cells, e.g., from which the cancer originated, from the same subject, and/or from a different subject or subjects. This disclosure is based at least in part on a series of studies on cellular ECVs from rats, mice, and humans. Both reversal and acquisition of chemotherapy resistance and anchorage-independent growth (a hallmark of cellular transformation) using models of prostate and breast cancer cell lines exposed to highly purified preparations of ECVs from non-malignant and malignant breast and prostate cell lines have been demonstrated. ECVs isolated from normal rat liver perfusions strongly suppress anchorage-independent growth of established rat liver hepatocellular carcinoma (HCC) cell lines. These data provide evidence that cancer is a disease caused not solely by genetic mutations, but also one of cellular miscommunication. Therapeutic approaches to fighting disseminated cancers, via the infusion of non-cancerous cell ECVs, are provided.

### Methods of Treating Cancer

ECVs from non-cancerous cells can be used to transform cancerous cells to reverse drug resistance and sensitize cancerous cells to chemotherapy. In breast cancer, applicants have demonstrated that resistance of cells to doxorubicin can be reversed via ECVs, while in prostate cancer, resistance of cells to campothecin can be reversed via ECVs, as measured by apoptosis, cytotoxicity, and growth in soft agar.

Accordingly, methods of treating cancer, e.g., prostate cancer, breast cancer, lung cancer, colon cancer, kidney cancer, liver cancer, brain cancer, leukemia, lymphoma, multiple myeloma, pancreatic cancer, renal cell carcinoma, stomach cancer, bone cancer, hematological cancer, neural tissue cancer, melanoma, thyroid cancer, ovarian cancer, testicular cancer, cervical cancer, vaginal cancer, bladder cancer, carcinoma, sarcoma, metastatic disorders, and/or hematopoietic neoplastic disorders are provided herein. The methods can involve diagnosing a subject, preparing ECVs from non-cancerous cells, administering to a subject, having or at risk for developing cancer, a therapeutically effective amount of non-cancerous cell ECVs, wherein the non-cancerous cell ECVs are ECVs of non-cancerous prostate, breast, lung, colon, kidney, liver, brain, pancreas, stomach, bone, thyroid, ovary, testicle, cervical, vaginal, and/or bladder cells. The subject can be further monitored for treatment response.

Non-cancerous cell ECVs can be administered, e.g., by injection or infusion, to the subject. For example, non-cancerous cell ECVs can injected directly into or around the cancerous tissue. Non-cancerous cell ECVs can also be administered to bathe, e.g., cover, a cancerous tissue or organ to expose the cancerous tissue or organ to the non-cancerous cell ECVs. Skilled practitioners will appreciate that the non-cancerous cell ECVs can be administered to a subject at a concentration of at least 20 μg/kg, e.g., 30 μg/kg, 40 μg/kg, 50 μg/kg, 60 μg/kg, 70 μg/kg, 80 μg/kg, 90 μg/kg, 100 μg/kg, 120 μg/kg, 140 μg/kg, 160 μg/kg, 180 μg/kg, 200 μg/kg, 220 μg/kg, 250 μg/kg, or 500 μg/kg.

Non-cancerous cell ECVs can be from non-cancerous cells of the same or different type as the cancerous cells to be treated in the subject. For example, non-cancerous cell ECVs from prostate cells can be used to treat prostate cancer. Similarly, non-cancerous cell ECVs from breast cells can be used to treat breast cancer; non-cancerous cell ECVs from lung cells can be used to treat lung cancer; non-cancerous cell ECVs from colon cells can be used to treat colon cancer; non-cancerous cell ECVs from kidney cells can be used to treat kidney cancer; non-cancerous cell ECVs from liver cells can be used to treat liver cancer; and non-cancerous cell ECVs from brain cells can be used to treat brain cancer.

In any of the methods described herein, the subject may be suspected of having, is at risk of having, or has cancer, e.g., prostate cancer, breast cancer, lung cancer, colon cancer, kidney cancer, liver cancer, brain cancer, leukemia, lymphoma, multiple myeloma, pancreatic cancer, renal cell carcinoma, stomach cancer, bone cancer, hematological cancer, neural tissue cancer, melanoma, thyroid cancer, ovarian cancer, testicular cancer, cervical cancer, vaginal cancer, bladder cancer, carcinoma, sarcoma, metastatic disorders, and/or hematopoietic neoplastic disorders.

The term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art-recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

A metastatic tumor can arise from a multitude of primary tumor types, including but not limited to those of prostate, breast, colon, lung, bone, and liver origin. Metastases develop, e.g., when tumor cells shed from a primary tumor adhere to vascular endothelium, penetrate into surrounding tissues, and grow to form independent tumors at sites separate from a primary tumor.

Cancers that may be treated using the methods and compositions of the present invention include, for example, cancers of the stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck and throat; Hodgkins disease, non-Hodgkins leukemia, sarcomas, choriocarcinoma, and lymphoma, among others.

A subject that is “suspected of having cancer” is one having one or more symptoms of the condition. Symptoms of cancer vary greatly and are well-known to those of skill in the art and include, without limitation, breast lumps, nipple changes, breast cysts, breast pain, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, or pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, pancreas metastases, difficulty swallowing, and the like.

A subject that is “at risk of having cancer” is one that has a predisposition to develop cancer (i.e., a genetic predisposition develop cancer such as a mutation in a tumor suppressor gene, e.g., BRCA1, p53, RB, or APC) or has been exposed to conditions that can result in cancer. Thus, a subject that is “at risk of having cancer” can be one that has been exposed to mutagenic or carcinogenic levels of certain compounds (e.g., carcinogenic compounds in cigarette smoke such as acrolein, arsenic, benzene, benz {a}anthracene, benzo{a}pyrene, polonium-210 (Radon), urethane, or vinyl chloride). Moreover, the subject can be “at risk of having cancer” when the subject has been exposed to, e.g., large doses of ultraviolet light or X-irradiation, or exposed (e.g., infected) to a tumor-causing/associated virus such as papillomavirus, Epstein-Barr virus, hepatitis B virus, or human T-cell leukemia-lymphoma virus. Cancers are frequently treated with any of a variety of chemotherapeutic agents, which can be administered in conjunction with ECVs.

Subjects considered at risk for developing cancer may benefit particularly from the invention, primarily because prophylactic treatment can begin before there is any evidence of the disorder. Subjects “at risk” include, e.g., subjects exposed to carcinogens, e.g., by consumption, e.g., by inhalation and/or ingestion, at levels that have been shown statistically to promote cancer in susceptible subjects. Also included are subjects at risk due to exposure to ultraviolet radiation, or their environment, occupation, and/or heredity, as well as those who show signs of a precancerous condition such as polyps. Similarly, subjects in very early stages of cancer or development of metastases (i.e., only one or a few aberrant cells are present in the subject's body or at a particular site in a subject's tissue) may benefit from such prophylactic treatment.

Skilled practitioners will appreciate that a subject can be diagnosed by a physician (or veterinarian, as appropriate for the subject being diagnosed) as suffering from or at risk for a condition described herein, e.g., cancer, by any method known in the art, e.g., by assessing a subject's medical history, performing diagnostic tests, and/or by employing imaging techniques.

Skilled practitioners will also appreciate that ECV compositions need not be administered to a subject by the same individual who diagnosed the subject (or prescribed the ECV composition for the subject). ECV compositions can be administered (and/or administration can be supervised), e.g., by the diagnosing and/or prescribing individual, and/or any other individual, including the subject her/himself (e.g., where the subject is capable of self-administration).

Common chemotherapeutic agents and/or treatments can be used in conjunction with ECVs to treat cancer. For example, campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, methotrexate, and an analog of any of the aforementioned.

### Pharmaceutical Compositions

Also described herein are pharmaceutical compositions, which include a substantially pure preparation of non-cancerous cell ECVs and a pharmaceutically acceptable carrier. The non-cancerous cell ECVs can be isolated or prepared by known methods from non-cancerous cells, e.g., prostate, breast, lung, colon, kidney, liver, and/or brain cells, e.g., where the ECVs are substantially free of cells and media, e.g., at least or about 70%, at least or about 75%, at least or about 80%, at least or about 85%, at least or about 90%, at least or about 92%, at least or about 95%, at least or about 96%, at least or about 97%, at least or about 98%, or at least or about 99% purified. For example, ECVs can be isolated from conditioned medium after two to seven days of culture (Renzulli et al., J Urol 184:2165-2171, 2010). In one useful exemplary method, ECVs can be isolated from conditioned medium by a series of centrifugations. The resulting pellet is resuspended in PBS and an equal volume of the supravital red fluorescent cell membrane dye PKH26, diluted 1:250 in diluent C (Sigma®) and the cell cytoplasm supravital dye CFSE (Invitrogen) at a final concentration of 0.02 μM. ECVs are then incubated for 15 minutes at 37° C. An equal volume of 10% fetal bovine serum solution in PBS is then added and samples are ultracentrifuged at 28,000×gravity for one hour at 4° C. to yield a preparation of cell ECVs.

The pharmaceutical compositions can include nanoparticles, e.g., nanoparticles with a diameter range between 1 to 2500 nanometers, e.g., 1 to 2000 nanometers, 1 to 1500 nanometers, 1 to 1000 nanometers, 1 to 500 nanometers, 1 to 250 nanometers, 1 to 200 nanometers, 1 to 150 nanometers, 1 and 100 nanometers, 1 to 50 nanometers, and 1 to 25 nanometers. For example, isolated non-cancerous cell ECVs can be incorporated in a nanoparticle or on a nanoparticle. Skilled practitioners would recognize art-known methods to incorporate an ECV into a nanoparticle or on a nanoparticle.

A “pharmaceutically acceptable carrier” can include solvents, e.g., saline, dispersion media, coatings, antibacterial and antifungal agents, isotonic and absorption delaying agents, and the like, compatible with pharmaceutical administration. Supplementary active compounds can also be incorporated into the compositions. The pharmaceutical compositions can be included in a container, pack, or dispenser together with instructions for administration.

A pharmaceutical composition is formulated to be compatible with its intended route of administration. Examples of routes of administration include direct injection, infusion, bathing, exposure, parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Skilled practitioners will appreciate that the pharmaceutical compositions described herein contain a concentration of ECVs that would enable convenient administration of the composition to a subject at a concentration of at least 20 μg/kg, e.g., 30 μg/kg, 40 μg/kg, 50 μg/kg, 60 μg/kg, 70 μg/kg, 80 μg/kg, 90 μg/kg, 100 μg/kg, 120 μg/kg, 140 μg/kg, 160 μg/kg, 180 μg/kg, 200 μg/kg, 220 μg/kg, 250 μg/kg, or 500 μg/kg.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, a polyol (for example, glycerol, propylene glycol, and liquid polyethylene glycol, and the like), or a suitable mixture thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent that delays absorption, e.g., aluminum monostearate or gelatin, in the composition.

Sterile injectable solutions can be prepared by incorporating the ECVs in the required amount in an appropriate solvent with one or a combination of ingredients enumerated above, as required, followed by filtered sterilization. Typically, dispersions are prepared by incorporating the ECVs into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Injectable compositions may contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like). For intravenous injections, the ECVs may be administered by the drip method, whereby a pharmaceutical composition containing the ECVs and a physiologically acceptable excipient is infused. Physiologically acceptable excipients may include, for example, 5% dextrose, 0.9% saline, Ringer's solution or other suitable excipients. For intramuscular preparations, a sterile composition of a suitable soluble salt form of the compound can be dissolved and administered in a pharmaceutical excipient such as Water-for-Injection, 0.9% saline, or 5% glucose solution, or depot forms of the compounds (e.g., decanoate, palmitate, undecylenic, enanthate) can be dissolved in sesame oil.

Oral compositions typically include an inert diluent or an edible carrier. For the purpose of oral therapeutic administration, the ECVs can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring. Alternatively, the pharmaceutical composition can be formulated as a chewing gum, lollipop, or the like.

Liquid compositions for oral administration prepared in water or other aqueous vehicles can include solutions, emulsions, syrups, and elixirs containing, together with the ECVs, wetting agents, sweeteners, coloring agents, and flavoring agents. Various liquid and powder compositions can be prepared by conventional methods for inhalation into the lungs of the patient to be treated.

For administration by inhalation, the ECVs are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the ECVs are formulated into ointments, salves, gels, or creams as known in the art.

The compositions can also be prepared in the form of suppositories (e.g., with conventional suppository bases such as cocoa butter and other glycerides) or retention enemas for rectal delivery.

In one embodiment, the ECVs are prepared with carriers that will protect the ECVs against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of ECVs calculated to produce the desired therapeutic effect in association with the required pharmaceutical carrier.

In all of the methods described herein, appropriate dosages of ECVs can readily be determined by those of ordinary skill in the art of medicine, e.g., by monitoring the patient for signs of disease amelioration or inhibition, and increasing or decreasing the dosage and/or frequency of treatment as desired. Toxicity and therapeutic efficacy of such ECVs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Care should be taken to design a delivery system that targets ECVs to the site of affected tissue, e.g., prostate gland or breast can be employed, to minimize potential damage to non-cancerous cells and, thereby, reduce any potential side effects.

The data obtained from cell culture assays and animal studies can be used in formulating a range of dosage for use in humans. The dosage of ECVs lies preferably within a range of circulating concentrations that include the ED50 with little or no toxicity. The dosage may vary within this range depending upon the dosage form employed and the route of administration utilized. For any ECV used in the method of the invention, the therapeutically effective dose can be estimated initially from cell culture assays. A dose may be formulated in animal models to achieve a circulating plasma concentration range that includes the IC50 (i.e., the concentration of the test ECV which achieves a half-maximal inhibition of symptoms) as determined in cell culture. Such information can be used to more accurately determine useful doses in humans. Levels in plasma may be measured, for example, by high performance liquid chromatography.

The ECVs can be administered one time per day, twice per day, one time per week, twice per week, for between about 1 to 52 weeks per year, e.g., between 2 to 50 weeks, about 6 to 40 weeks, or for about 4, 5, or 6 weeks. The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of ECVs can include a single treatment or, preferably, can include a series of treatments.

### EXAMPLES

The invention is further described in the following examples, which do not limit the scope of the invention described in the claims.

Epigenetic mechanisms of gene regulation collaborate with genetic alterations during cancer development. This is evident from every aspect of tumor biology including cell growth and differentiation, cell cycle control, DNA repair, angiogenesis, and migration. In contrast to the genetic factors that promote cancer, other factors can reverse the cancer phenotype and potentially may provide new targets for therapeutic intervention.

Recent attention has been focused on the task of identifying soluble factors secreted by tumor cells that are responsible for epigenetic changes during cancer development. In addition to soluble paracrine factors, many tumor cells also release ECVs. Interestingly, the abundance of ECVs released generally correlates positively with advanced grade and stage of cancer progression. However, the mechanism triggering ECV generation by cancer cells is unknown. Nonetheless, there is mounting evidence that vesicle trafficking is a highly important process in cancer progression.

Biopsied prostate tumor cells co-cultured with human bone marrow (BM) cells induce expression of prostate specific genes (Renzulli et al., J Urol 184:2165-71, 2010). Significantly, it is possible to reverse drug resistance and sensitize nonmalignant and malignant breast and prostate cells to chemotherapy-inducing effects of clinically relevant agents via ECVs. The fact that ECVs can elicit epigenetic changes presents an opportunity for the use of therapeutic agents to A) block ECV release from cancer cells, e.g., using an antibody to an intracellular protein or a cell surface receptor and/or a chemical inhibitor; B) identify genetic material and/or agents which may block non-malignant cells from accepting the ECVs; and C) accelerate the transfer of genetic material from driver ECVs to passenger malignant cells to inhibit tumor progression.

Changes in cancer and most importantly changes when cancer cells revert to a non-malignant phenotype are due to ECV microenvironmental influences on the gene expression of the cancer cells, specifically on the transcriptional mechanisms of that cell. The molecular studies in progress will define whether epigenetic changes are the cause of the phenotype change, i.e., driver events, or not, i.e., passenger events. The testing of this hypothesis is important for the field in that it would 1) indicate a new mechanism for alterations of the epigenetic profile of the cancer cells and 2) provides a unique new approach to the therapy of cancer; the infusion of non-malignant cell ECVs.

Murine lung ECVs enter murine marrow cells and epigenetically change their phenotype towards that of lung cells (Aliotta et al., Stem Cells 25:2245-56, 2007; Aliotta et al., Exp Hematol 38:233-45, 2010). ECVs were prepared by ultracentrifugation (100,000×gravity) of lung-conditioned media, stained the ECV laden pellet with the fluorescent dyes CFSE (green) and PKH26 (red) and then separated the ECVs as red/green events by fluorescent activated cell sorting (FACS). When these purified ECVs were incubated with murine marrow cells, only the cells taking up the ECVs evidenced expression of the lung specific mRNA for Surfactants A-D, clara cell protein and aquaporin 5. The lung ECVs contained many species of mRNA, microRNA, protein and some DNA (mitochondrial and genomic). Marrow cells exposed to lung ECVs express lung-specific mRNA and protein and showed an increased capacity to convert to type 2 pneumocytes after in vivo transplantation into lethally irradiated mice. The expression of lung specific mRNA immediately after ECV exposure is due to the direct delivery of lung-specific mRNA and to the delivery of a separate transcription factor(s). However, expression of lung-specific mRNA persists out to 12 weeks of cytokine supported in vitro liquid culture and this long-term expression is due to transcriptional activation of target marrow cell DNA (Aliotta et al., Exp Hematol 38:233-45, 2010). These latter experiments involved rat lung ECVs exposed to mouse marrow and determination, using species-specific primers, of whether surfactants Band C were of rat or mouse origin. With long-term expression in culture of surfactants B and C by mouse marrow cells exposed to rat ECVs, the mRNA was all mouse. Thus the exposure of murine marrow to lung ECVs resulted in a stable long-term epigenetic change toward the genetic phenotype of lung cells. In ongoing studies, tissue-specific expression of mRNA was found when marrow cells were exposed to ECVs from murine liver, heart, and brain. Further, human explant lung cancer fragments or prostate cancer fragments induce tissue specific changes in human marrow cells. Altogether, these observations show that there is robust and stable modulation of cellular phenotype through vesicle cell entry and those cells can be altered toward the phenotype of the cell type giving rise to the ECV.

Specific proteins are transferred from malignant chemotherapy resistant breast cancer cells to normal cells, implying that these proteins may affect the acquisition of the malignant phenotype in this model. Recent studies have directly addressed the potential of vesicles from non-malignant human cells to alter the phenotype of cancer cells. Vesicles from prostate or breast non-malignant tissue reversed anchorage-independent growth and/or chemo resistance of prostate and breast cancer cell line cells and of cells from tissue from prostate surgeries. These observations then need to be considered in the light an alternate theory of carcinogenesis termed the tissue organization field theory (Soto et al., Bioessays 26:1097-107, 2004; Sonnenschein et al., Semin Cancer Biol 18:372-7, 2008) that proposes that cancer, rather than being caused by specific gene mutations, is a problem of tissue organization, comparable to organogenesis during early development.

### Experimental Procedures

Materials

All reagents and chemicals were purchased from Sigma Chemical Co. (St. Louis, Mo.) unless otherwise noted. ST2461, a CPT analog, was provided by Sigma Tau (Rome, Italy). Protein quantification reagents were obtained from Bio-Rad Laboratories, Inc. (Hercules, Calif.). Enhanced chemiluminescence reagents and secondary mouse and rabbit horseradish peroxidase-conjugated antibodies for Western blot analysis were ordered from GE Healthcare (Arlington Heights, Ill.). Antibodies to RKIP were purchased from Millipore (Hopkington, Mass.); antibodies to actin-HRP, pRKIP, SOCS2, prohibitin, 14-3-3, and STAT3 were obtained from Santa Cruz Biotechnology (Santa Cruz, Calif.). PARP antibodies were purchased from Invitrogen (Carlsbad, Calif.).

Cells

The human prostate carcinoma cell line DU145 was obtained from the American Type Culture Collection (ATCC) (Rockville, Md.). The RC1 cell line, which was derived from DU145 cells, has been described (Chatterjee et al., J Biol Chem 279:17515-17523, 2004). Cell lines were used between passage numbers 10-20. Cells were grown in RPMI 1640 medium and supplemented with 10% fetal calf serum, glutamine, non-essential amino acids, 100 units/ml penicillin and 50 units/ml streptomycin and cultured in a humidified incubator at 37° C. containing 5% CO₂. The prostate epithelial cells (PrECs) utilized in this study were obtained from Dr. William Hahn at Dana Farber Cancer Institute, Boston, Mass. (Berger et al., Cancer Res 64:8867-8875, 2004) and gown in PrEBM media supplemented with PrEGM SingleQuots (Lonza, Walkersville, Md.). PrECs were used in between passage 2 and 10.

Western Blot Analysis

Total cell extracts were prepared as previously described, and protein concentrations of lysates were determined using a Bradford assay kit (BioRad). Proteins were separated by SDS-PAGE and electrophoretically transferred from the gel to nitrocellulose membranes (GE Healthcare). Proteins recognized by antibodies were detected by enhanced chemilluminescence reagents (GE Healthcare).

ECV Isolation

ECV isolation was conducted for two different patient cancer tissue samples, as well as for the DU145 and PrEC cell lines. Tumor samples were weighed and minced with a sterile scalpel into 1 to 2 cm pieces as previously reported (Renzulli et al., J Urol 184, 2165-2171, 2010). Tissue pieces were then subjected to enzymatic dissociation using 0.2% collagenase in DMEM with 10% FBS for 90 minutes at 37° C. and passed sequentially through 18, 22, and 25 gauge needles followed by a 40 μm cell strainer. The cell suspension was washed twice with DMEM and plated into a T-75 tissue culture flask with growth media consisting of DMEM 10% ECV-free FBS, 1% penicillin-streptomycin. DU-145 cells were plated at 1.5×10⁵ cells per T75 flask, and PrECs were plated at 1.5×10⁶ cells per 100 mm plate. Cell cultures were maintained under the previously listed conditions, and after seven days of culture, or approximately five doublings of normal and tumor tissue as previously reported, the conditioned medium (CM), the media from the cultured cells, was removed and further processed to isolate ECVs (Renzulli et al., J Urol 184, 2165-2171, 2010).

CM for ECV isolation was centrifuged at 300×gravity for 10 minutes at 4° C. Supernatant was ultra-centrifuged (UCF) at 24,000×g for one hour at 4° C. The UCF pellet was re-suspended in growth medium and co-cultured (self-culture or cross-culture) with cells for 4 to 7 days. The UCF pellet was further processed for co-culture (see below) or the isolation of protein for phospho-protein (Kinexus) and mass spectrometry analysis.

Co-Culture of Prostate Tissue Extracellular Vesicles with Non- and Malignant Prostate Epithelial Cell Lines

Non-malignant human PrECs and malignant DU145 prostate cells were grown in Lonza Bullet or RPMI medium, respectively, supplemented with special additives (PrEC) or 10% dialyzed ECV-free FBS and antibiotics (DU145). The cells were co-cultured with ECVs from normal or malignant prostate tissue. Specifically, PrECs were co-cultured with ECVs from prostate tumor tissue, and malignant DU145 cells were co-cultured with ECVs from normal prostate tissue.

Soft Agar Cloning

Following ECV co-culture, cells were grown in normal growth medium. After seven days, cells were harvested for soft agar colony formation. The lower layer of the dish contained 2 ml of 1% agarose mixed with growth media; on the top level, 0.4% agarose mixed with growth media and 0.05-1×10⁵ cells to a final volume of 1 ml. Plates were incubated in 5% CO₂ at 37° C. for 2 to 3 weeks. Colonies were then counted and images were captured on an Olympus MT2 microscope. Each experiment was analyzed in quadruplicate by three different individuals.

Kinexus Array Analysis

Extracellular vesicles were harvested as previously discussed but co-culturing was conducted with 1×10⁶ cells on 100 mm plates. Cells were harvested washed twice with PBS, and centrifuged at 14,000×g for 5 minutes. Supernatant was discarded and the resulting pellet was frozen at −20° C. Pellets were sent to Kinexus Bioinformatics (Vancouver, British Columbia, Canada) for proteomic analysis.

Tissue Collection

Fresh prostate tissue specimens were obtained following robotic assisted laparoscopic prostatectomy (Da Vinci Robotic Surgical System at The Miriam Hospital). In accordance with the requirements mandated by the Department of Health, the active robotic surgeons have maintained a detailed database with preoperative, intraoperative, and postoperative parameters recorded for each case. ECVs were isolated from two patients. Patient 18: Gleason 4+4=8 with tertiary 5 pattern (high risk) prostate cancer with positive margin focally and seminal vesicle invasion (pathology stage T3b). Patient 19: Gleason 4+4=8 with tertiary 5 pattern (high risk) prostate cancer with negative margins (pathology stage T3a). Patients 13, 14, and 16 were also Gleason grade 8. Tissue was processed and ECVs isolated as previously described (Renzulli et al., J Urol 184, 2165-2171, 2010).

Protein Extraction

Protein lysates from PrECs that were co-cultured with patient ECVs for seven days and control PrECs co-cultured with PrEC ECVs were obtained using a ReadyPrep Sequential Extraction Kit (Bio-Rad) and then the sequential extractions were combined and cleaned up using a ReadyPrep 2-D Clean-Up Kit (Bio-Rad). Total protein concentration was determined using a BCA protein assay kit (Thermo Scientific). Samples were then resolved using NuPAGE SDS-PAGE system (Invitrogen) (4-12% acrylamide, Bis-Tris with MES SDS Running Buffer) and stained with Gel Code Blue Stain (Thermo Scientific). Gel lanes corresponding to each sample were excised into three bands that covered regions of high, medium, and low molecular weight proteins to reduce sample complexity. Each band was cut into 6 mm wide pieces and subjected to in-gel tryptic digestion, and then each fraction was washed/dehydrated twice in a 1:1 solution of 0.1 M ammonium bicarbonate (Sigma) and 100% ACN (Sigma). Disulfide bonds were reduced with 10 mM dithiothreitol (DTT)(Thermo)/0.1 M ammonium bicarbonate for 45 minutes at 56° C. and alkylated with 55 mM iodoacetamide (IAA)(Sigma) for 30 minutes at room temperature in the dark, and washed/dehydrated twice as explained above followed by trypsin digestion overnight at 37° C. After trypsin digestion, peptides were extracted using 25 mM ammonium bicarbonate and 100% ACN, followed by two rounds of 5% formic acid and 100% ACN. The extracts were pooled, dried in a vacuum centrifuge, and stored at −20° C. until LC/MS analysis.

Liquid Chromatography/MS Analysis of Protein Digests

Mass spectrometry analysis was performed at the Rhode Island Hospital Proteomics Core facility by nano-LC-ESI-MS/MS using an Ultimate3000 nano-LC system (Dionex) controlled with Chromeleon software coupled to a QSTAR XL (Applied Biosystems, Concord, Ontario, CA) mass spectrometer. Tryptic digests were fractionated by reversed-phase chromatography using a C-18 PepMap 100 column (75 μm id×15 mm, 3 μm particle size, LC Packings/Dionex, Sunnyvale, Calif.) operating at a flow rate of 300 nL/min. A linear separation gradient applied was starting at 5% (v/v) ACN in 0.1% (v/v) formic acid (Buffer A) to 95% (v/v) ACN in 0.1% (v/v) formic acid (Buffer B) over a 40 minute gradient. The column eluate was introduced directly into the mass spectrometer via ESI.

Candidate ions were selected and fragmented using a standard information dependent acquisition (IDA) method. One second MS scans (range between 350 and 1800 Thompson, Thompson (Th)=Da/z) were used to identify candidates for fragmentation during MS/MS scans. MS/MS scans (2 s; range between 150 and 1800 Th) were collected up to three times after each survey scan. In order for an ion to be considered a candidate for fragmentation, it had to be assigned a charge in the range of +2 to +4.

Data Processing for Protein Identification and Quantitation

Raw LC-MS/MS data were converted using ABSciex MS Data converter software (v1.3 beta) to mgf format for protein identification using MASCOT v2.3.2 search engine (Matrix Science, Boston, Mass., USA) by searching against a non-redundant human UniProt database (Apr. 20, 2012, containing 87,656 protein entries) using the following parameters: tryptic peptides with up to two missed cleavage sites, peptide tolerance of 0.2 Da, fragment tolerance of 0.5 Da, instrument type: ESI-QUAD-TOF, and variable modifications: methionine oxidation.

For label-free protein quantitation and proteome comparisons, raw files were converted to mzXML format using ABSciex MS Data converter software (v1.3 beta) and uploaded along with Mascot search results in .dat format into ProteolQ software (v.2.3.08 BIOINQUIRE, Athens, Ga.). Spectral counting and relative intensity quantification were performed using precursor ion intensities, with the following parameters: mass tolerance of 20 ppm, minimum peptide length of six amino acids, protein probability of 0.5, and peptide probability of 0.05. After protein set generation, the proteins were further filtered using a 0.9 protein probability and normalized according to the number of spectra in each sample. Then the proteins that the five Gleason grade 8 patients had in common were placed in a new protein set and were filtered using GO annotations which describe the role of a given gene in a biological process, its molecular function, and cellular component. The GO terms which were selected to filter the results are related to apoptosis, inflammation, immune response, DNA transcription, and DNA translation in order to determine the importance and behavior of these proteins in apoptotic and cell survival pathways.

Ingenuity Pathway Analysis (IPA) (Ingenuity Systems, Redwood City, Calif., USA) was used to identify protein networks according to biological functions and/or diseases in the Ingenuity Pathway Knowledge Base (IPKB). The protein accession numbers and the corresponding log₂ relative expression values were uploaded into IPA, where the log₂ relative expression values are converted to fold change values by the software. Using these fold change values (with a cutoff of 1.5 for up- or down-regulation) for each protein, IPA determines the statistically relevant (p<0.05) canonical pathways and functions related to the proteins in each sample. Each pathway and function is assigned a −log(p-value) which is determined by the number of proteins present in the specific pathway or function and the statistical significance of the expression level of the protein.

Statistical Methods

All cell culture experiments were repeated at least thre times, unless indicated otherwise, and paired t-tests were used to determine statistical significance.

### Example 1

### ECV-Mediated Reversal of Drug Resistance in Prostate Cancer

Chemotherapy is currently the major treatment option for castration-resistance prostate cancer. However, chemoresistance is inherent in half of all patients that receive chemotherapy, and the decline of sensitivity to therapeutic agents in patients that initially respond is inevitable (Mahon et al., Endocr Relat Cancer 18:R103-123, 2011). Multiple cellular pathways involving apoptosis, inflammation, angiogenesis, signaling intermediaries, drug efflux pumps, and tubulin are implicated in the development of chemoresistance (Mahon et al., Endocr Relat Cancer 18:R103-123, 2011). It has been shown that resistance to CPT in DU145 cells is due, in part, to expression of Raf kinase inhibitor protein (RKIP) (Chatterjee et al., J Biol Chem 279:17515-17523, 2004). In addition to RKIP, resistance to CPT is due to the release of ECVs.

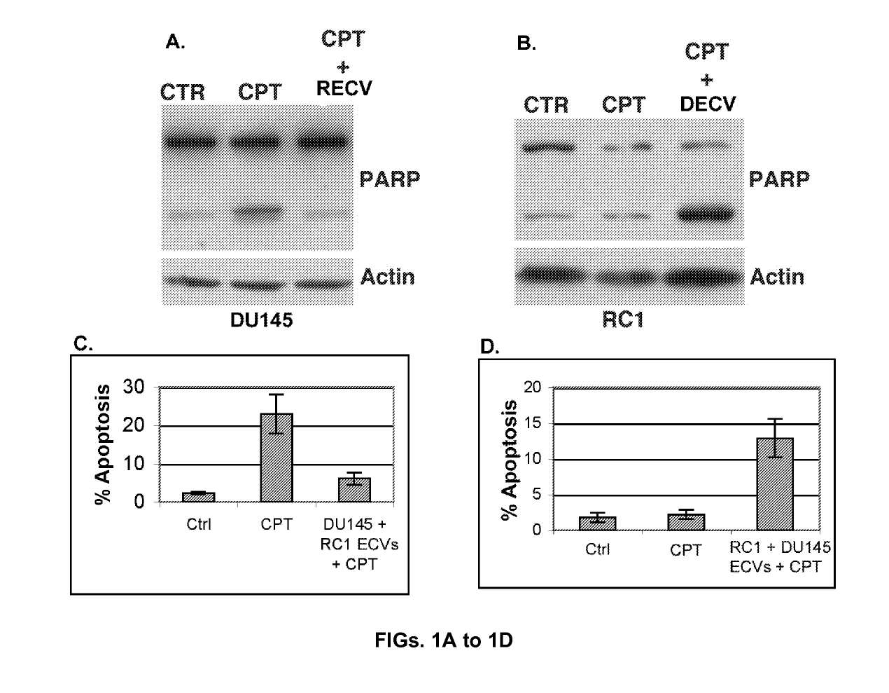

To investigate the effects of ECV-mediated transfer of chemoresistance in prostate cancer, the mechanism of resistance to camptothecin (CPT) was studied in human prostate cancer cell lines. The DU145 cell line, a human prostate carcinoma cell line, undergoes extensive apoptosis when treated with 9-nitrocamptothecin (9NC) (Chatterjee et al., J Biol Chem 279:17515-17523, 2004). CPT inhibits topoisomerase I, thereby inducing single-strand breaks into the DNA molecule (Covey et al., Cancer Res 49:5016-5022, 1989). Conditioned media from parental DU145 cells and DU145 cells resistant to CPT (RC1 cells) were collected, ultracentrifuged, and ECVs were collected for co-culture (Renzulli et al., J Urol 184:2165-2171, 2010). ECVs isolated from DU145 cells were co-cultured with RC1 cells and ECVs from RC1 cells were co-cultured with DU145 cells. After six days, both groups were treated with CPT, cells were harvested and analyzed for apoptosis via Poly ADP Ribose Polymerase (PARP) cleavage. Upon DNA damage, PARP signals DNA repair enzymes. Treatment of DU145 with CPT indicates an increase in PARP cleavage compared to untreated control cells (CTR) indicating PARP mediated activation of DNA repair while DU145 cells co-cultured with ECVs from RC1 cells demonstrated reduction in PARP cleavage similar to the control (FIGS. 1A and 1B). These results demonstrate ECV-mediated chemoresistance of DU145 cells. DU145 cells co-cultured with RC1 ECVs did not undergo apoptosis after CPT treatment, whereas the RC1 cells co-cultured with the DU145 ECVs were now sensitized to the apoptosis-inducing effects of CPT (FIGS. 1C and 1D). The same experiment was repeated, and the cells were analyzed for apoptosis via flow cytometry. As shown in FIG. 1C, DU145 cells became resistant to CPT-induced apoptosis after co-culture with RC1 ECVs. Conversely, RC1 cells underwent apoptosis upon treatment with CPT after being co-cultured with DU145 cell ECVs (FIG. 1D). These results indicate a phenotypic shift facilitated by the co-culturing with ECVs.

### Example 2

### ECV-Mediated Reversal of the Prostate Cancer Phenotype

To better understand the phenotypic switching capacity of ECVs, several strategies were applied. One of the hallmarks of malignant transformation of cells is the ability to exhibit anchorage-independent growth (Mori et al., Oncogene 28, 2796-2805, 2009). ECV-mediated phenotype changes were examined using a non-malignant model of prostate cancer in a malignant cell line to see if the phenotype can be transferred as measured by soft agar colony formation. ECVs were harvested from a malignant human prostate cancer cell line (DU145), as well as from an immortalized, non-tumorigenic prostate epithelial cell line (PrEC cells), and were collected for co-culture for vesicle characterization. ECVs isolated from DU145 cells were co-cultured with PrECs, and ECVs isolated from PrEC cells were co-cultured with DU145 cells. The number of ECVs used for co-culture was normalized by counting the total number of ECVs within a particular size of 30 to 1000 nm using the NANOSIGHT® NS500 (NanoSight, Wiltshire, United Kingdom).

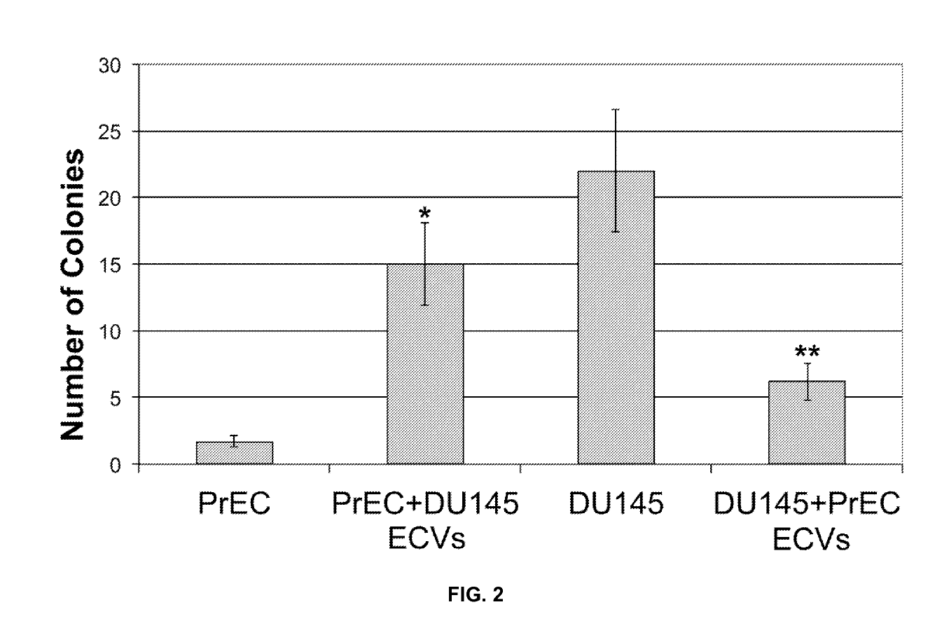

After three days in culture, the ability of each experimental condition (from above) to display anchorage-independent growth in soft agar was measured for 14 days. Since the malignant phenotype includes an increased ability to exhibit anchorage-independent growth, a significant increase or reduction in the number of colonies generated was viewed as a shift towards a tumorigenic phenotype or towards a normal phenotype, respectively. As shown in FIG. 2, co-culture of DU145 cells with ECVs isolated from PrECs inhibited colony formation in soft agar, indicating that anchorage-independent growth was significantly suppressed (p<0.0004) in comparison to DU145 cells without ECVs (FIG. 2). Remarkably, the reciprocal effect was also observed where significant changes in colony formation and anchorage independent growth in non-tumorigenic PrECs that were co-cultured with ECVs isolated from DU145 cells (p<0.00003) compared to PrECs without ECVs (FIG. 2).

Kinexus Proteomic Antibody Array Analysis of Proteins

To determine the proteins that are involved in or might be responsible for “phenotypic switching,” Kinexus phospho-protein microarray analysis (Kinexus Bioinformatics Corporation, Vancouver, BC) was utilized in the DU145 cells co-cultured with PrEC ECVs. ECVs were isolated from PrECs after seven days in culture as described (Renzulli et al., J Urol 184, 2165-2171, 2010). PrEC ECVs were then co-cultured with DU145 cells for seven days after which the cells were harvested and the pellet sent to Kinexus Bioinformatics Corporation for analysis. The ability of ECVs to elicit a phenotypic switch was therefore verified in the proteins that were transferred or induced and then analyzed. As a control, a sample with DU145 cells co-cultured with DU145 ECVs was also generated. Table 1 shows a portion of the results of the microarray analysis indicating the fold change in protein expression (Z score ratio).

TABLE 1
ECV-mediated transfer of proteins via Kinexus phospho-protein
microarray analysis in DU145 cells co-cultured with PrEC ECVs.

| Target Protein | |
|---|---|
| Name | Z-ratio (DuP, Du) |
| PKCz | 1.42 |
| SOCS2 | 1.30 |
| PKCm (PKD) | 1.05 |
| KAP | 1.05 |
| STAT5A | 1.04 |
| Cyclin G1 | 1.00 |
| IKKa | −1.01 |
| p38g MAPK | −1.02 |
| (Erk6) | |
| CK1g | −1.03 |
| MEK5 (MAP2K5) | −1.08 |
| CDK1 (CDC2) | −1.09 |
| STAT6 | −1.09 |
| IKKa | −1.14 |
| RIPK1 | −1.25 |
| STAT3 | −1.48 |
| PAK3 | −1.52 |
| RSK1 | −3.43 |

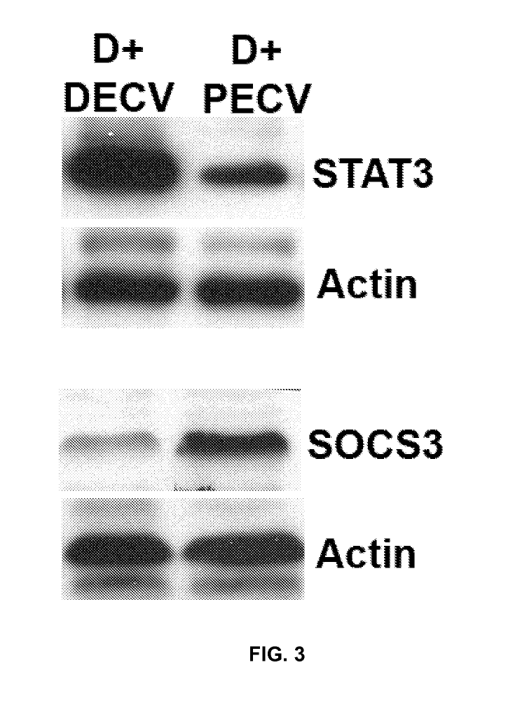

To further validate these results, the proteomic data was confirmed with Western blot analysis. Western blot analysis was performed with an aliquot of the sample that was retained in the lab prior to the shipment of the samples to Kinexus. A reduction in STAT3 expression in DU145 cells co-cultured with self-ECVs was observed, in comparison to DU145 cells co-cultured with PrEC ECVs. Further, there was a significant increase in SOCS2 expression in the DU145 cells co-cultured with PrEC ECVs (FIG. 3). This result implies that aberrant STAT3 signaling may be inhibited by ECV release and transfer of SOCS2 or regulators of STAT3 signaling network.

Mass Spectrometry Analysis of Prostate Cancer Patient ECVs

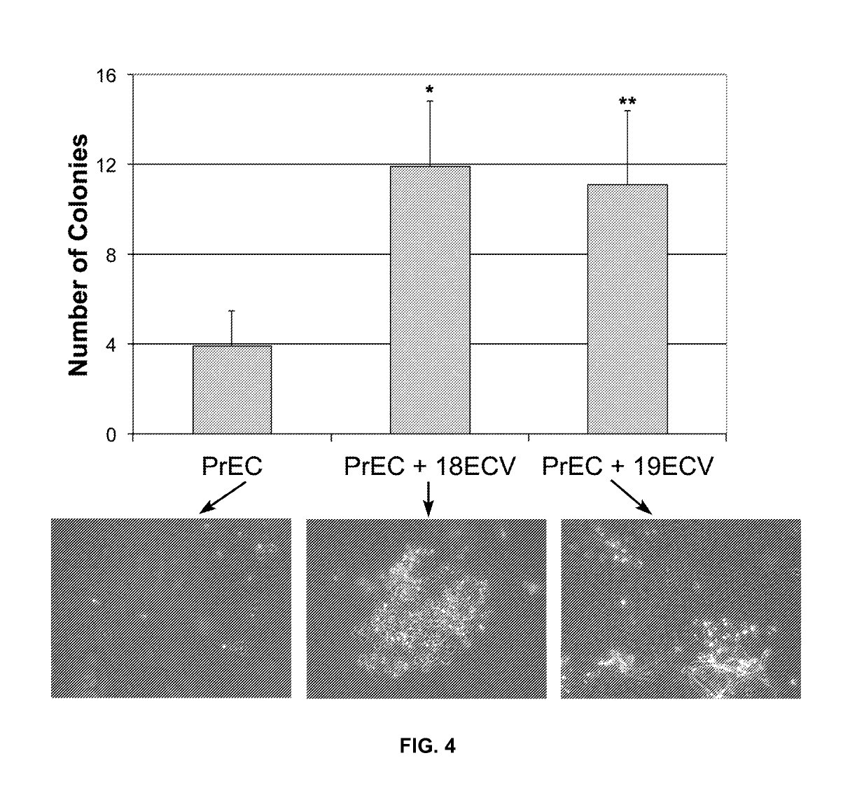

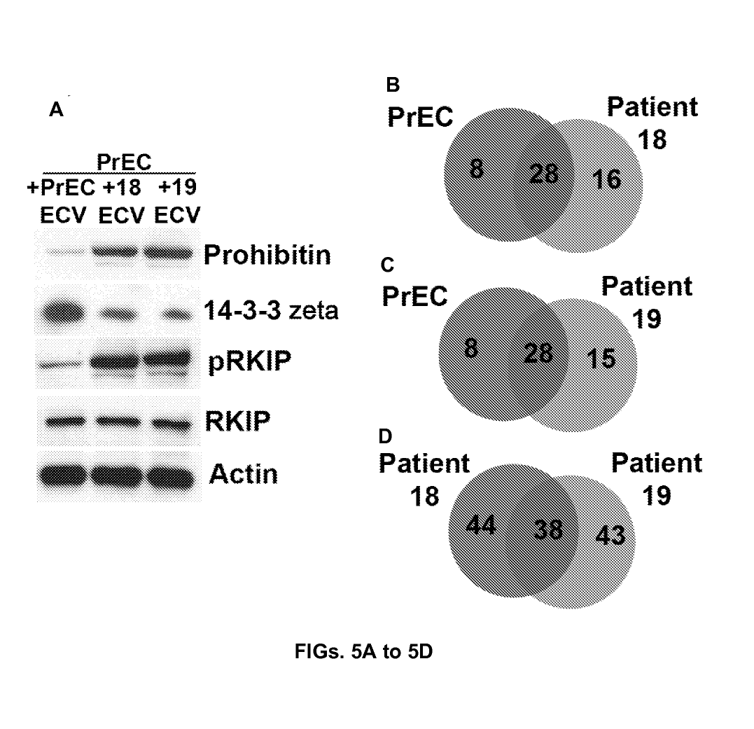

Studies on DU145 and PrEC ECVs and phenotype shifting were extended to ECVs from two prostate cancer patients, both with Gleason grade 8. Soft agar growth was measured in PrECs after co-culture with ECVs from prostate cancer patients 18 and 19. ECVs from patients 18 or 19 significantly increased soft agar growth in non-malignant PrECs (p<0.0000006 and p<0.00002, respectively) (FIG. 4). Note the increase in colony size in PrECs (bottom panel) co-cultured patient tumor ECVs. A portion of the sample used for soft agar cloning was analyzed by mass spectrometry. Table 2 shows a partial list of the proteins identified in PrECs exposed to tumor ECVs from patients 18 and 19 as well as the log₂ relative expression of each protein. Of note is the increase in RKIP when patient 18 and 19 ECVs were co-cultured with PrECs in reference to the levels of RKIP in PrECs alone. RKIP has been shown to regulate apoptosis and cell survival in prostate cancer (Chatterjee et al., J Biol Chem 279:17515-17523, 2004). Western blot analysis revealed that RKIP was phosphorylated after co-culture of patient 18 and 19 ECVs with PrECs (FIG. 5A). This result would explain, the data in FIG. 4 because pRKIP antagonizes the function of RKIP and allows for Raf/MAPK signaling to occur (Corbit et al., J Biol Chem 278:13061-13068, 2003). This pathway promotes oncogenesis and cell proliferation and, presumably, soft agar growth.

TABLE 2
Comparison of a sample of proteins that were transferred at higher
or lower relative expression from the patient ECVs to PrECs.

| Acces- | | PrEC vs | | PrEC vs | | PrEC + P18 vs | |
|---|---|---|---|---|---|---|---|
| sion | Protein | PrEC + P18 | | PrEC + P19 | | PrEC + P19 | |
| No. | Name | log₂ | # Pep | log₂ | # Pep | log₂ | # Pep |
| P06733 | Alpha-enolase | 0.051 | 5 | 0.044 | 2 | −0.007 | 2 |
| P04083 | Annexin A1 | 0.47 | 9 | 0.69 | 10 | 0.22 | 10 |
| P00403 | Cytochrome c | 0.86 | 4 | 0.82 | 4 | −0.04 | 4 |
| | oxidase subunit 2 | | | | | | |
| P68104 | Elongation | 0.04 | 8 | 0.39 | 8 | 0.35 | 8 |
| | Factor 1-alpha 1 | | | | | | |
| P30086 | PEBP1 (RKIP) | 1.12 | 5 | 1.88 | 4 | 0.75 | 4 |
| P35232 | Prohibitin | 0.08 | 2 | 0.41 | 2 | 0.33 | 2 |

In an analysis of the total proteome content of PrECs exposed to ECVs from patient 18, 36 protein groups were identified in PrECs alone and 44 protein groups were identified in PrECs with Patient 18 ECVs. From these, eight protein groups were unique to PrECs and 16 were unique in Patient 18 ECVs with 28 common protein groups (FIG. 5B). Exposure of PrECs with ECVs from Patient 19 yielded similar results (FIG. 5C). Analysis of proteome content between patients 18 and 19 yielded minimal differences between the numbers of protein groups identified in each sample indicating low patient heterogeneity (FIG. 5D).

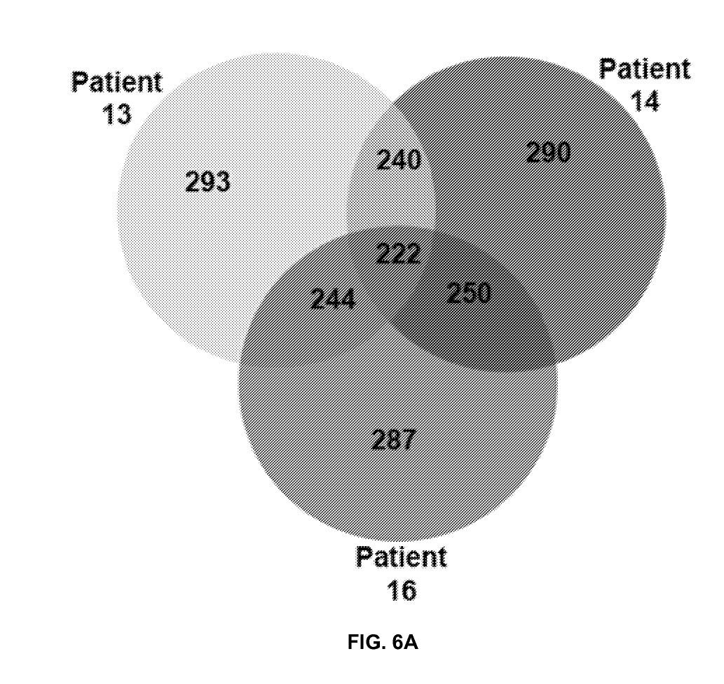

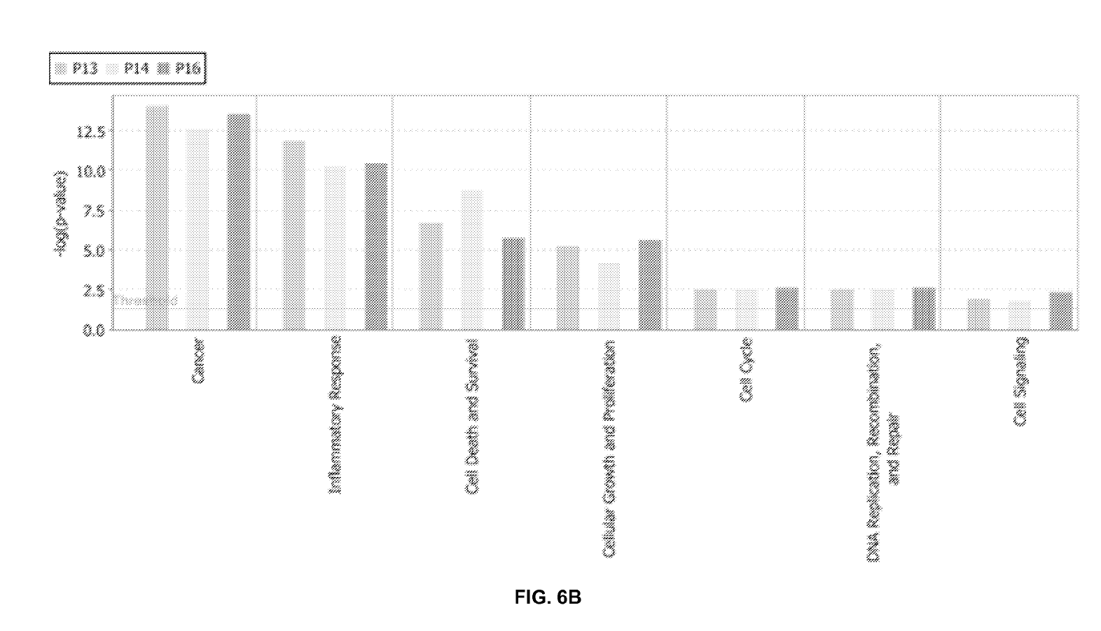

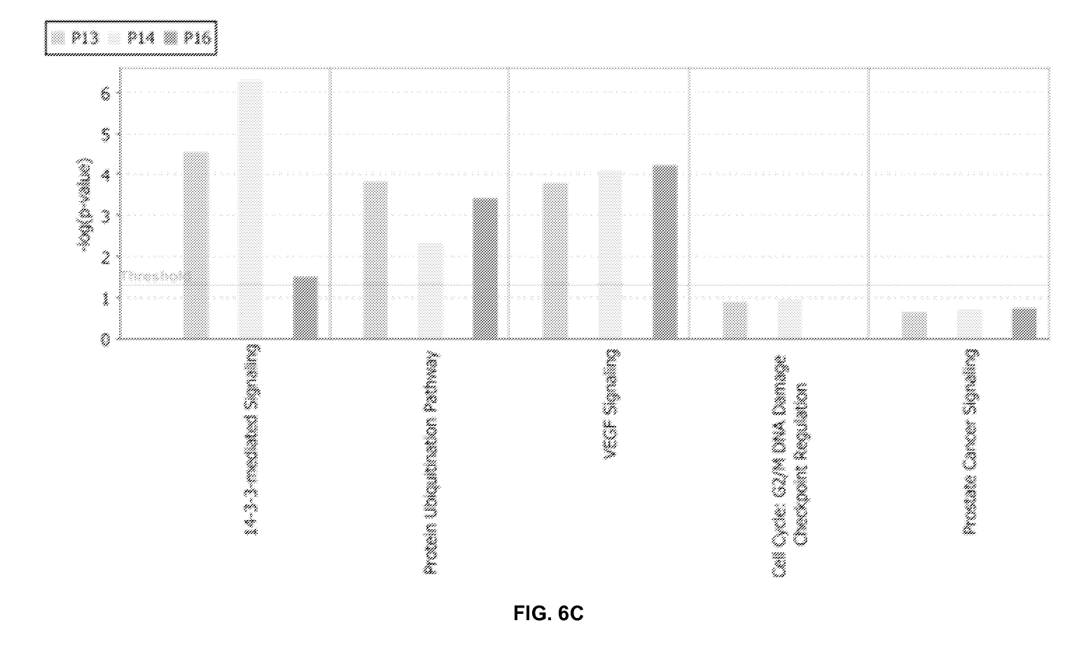

The ECV content of three additional Gleason grade 8 patients (Patients 13, 14, and 16) was examined (FIG. 6). The Venn diagram shows that there are 222 common proteins between these patients (FIG. 6A). The bar graph shows the functionalities listed by IPA based on the ProteolQ protein relative expression values (FIGS. 6B and 6C). The relative expression value of each protein in addition to the presence or absence of key proteins associated with that term both contribute to the significance value assigned to each function or pathway (indicated by the threshold line across the bar graph in FIGS. 6B and 6C).

In addition, patients 18 and 19 were compared with patients 13, 14, and 16 to determine common proteins between these five Gleason grade 8 patients. Seventy-one common proteins between the five Gleason grade 8 patients were then further filtered according to GO annotations which are related to apoptotic and cell survival pathways (seen in Tables 3, 4, 5, and 6). Proteins were found across all patients that are associated with apoptosis, growth and proliferation, inflammation, immune response, and DNA transcription and translation. This demonstrates that there is level of homogeneity in protein content among these five Gleason 8 patients, which provides a basis for targeting these proteins to improve therapeutic methods.

TABLE 3
Apoptosis related proteins found in the common proteins between
patients 13, 14, 16, 18, and 19.

| Accession # | Protein Name |
|---|---|
| P62258 | 14-3-3 protein epsilon |
| P63104 | 14-3-3 protein zeta/delta |
| P10809 | 60 kDa heat shock protein, mitochondrial |
| P11021 | 78 kDa glucose-regulated protein |
| O43707 | Alpha-actinin-4 |
| P04083 | Annexin A1 |
| P08758 | Annexin A5 |
| P27797 | Calreticulin |
| P14625 | Endoplasmin |
| P04406 | Glyceraldehyde-3-phosphate dehydrogenase |
| P32119 | Peroxiredoxin-2 |
| P02545 | Prelamin-A/C |
| P35232 | Prohibitin |
| P14618 | Pyruvate kinase isozymes M1/M2 |
| P08670 | Vimentin |
| P21796 | Voltage-dependent anion-selective channel protein 1 |
| P13010 | X-ray repair cross-complementing protein 5 |

TABLE 4
Inflammation and immune response related proteins found in the
common proteins between patients 13, 14, 16, 18, and 19.

| Accession # | Protein Name |
|---|---|
| P10809 | 60 kDa heat shock protein, mitochondrial |
| P04083 | Annexin A1 |
| P08238 | Heat shock protein HSP 90-beta |
| P32119 | Peroxiredoxin-2 |

TABLE 5
Growth and proliferation related proteins found in the common proteins
between patients 13, 14, 16, 18, and 19.

| Accession # | Protein Name |
|---|---|
| P62258 | 14-3-3 protein epsilon |
| Q04917 | 14-3-3 protein eta |
| P11021 | 78 kDa glucose-regulated protein |
| P04083 | Annexin A1 |
| P07355 | Annexin A2 |
| P06576 | ATP synthase subunit beta, mitochondrial |
| P27797 | Calreticulin |
| Q00610 | Clathrin heavy chain 1 |
| Q06830 | Peroxiredoxin-1 |
| P35232 | Prohibitin |
| P13010 | X-ray repair cross-complementing protein 5 |

TABLE 6
DNA transcription and translation related proteins found in the
common proteins between patients 13, 14, 16, 18, and 19.

| Accession # | Protein Name |
|---|---|
| Q04917 | 14-3-3 protein eta |
| P63104 | 14-3-3 protein zeta/delta |
| P06733 | Alpha-enolaxe |
| P27824 | Calnexin |
| P27797 | Calreticulin |
| P04843 | Dolichyl-diphosphooligosaccharide--protein |
| | glycosyltransferase subunit 1 |
| P68104 | Elongation factor 1-alpha 1 |
| P11142 | Heat shock cognate 71 kDa protein |
| P32119 | Peroxiredoxin-2 |
| P35232 | Prohibitin |
| P68371 | Tubulin beta-4B chain |
| P13010 | X-ray repair cross-complementing protein 5 |

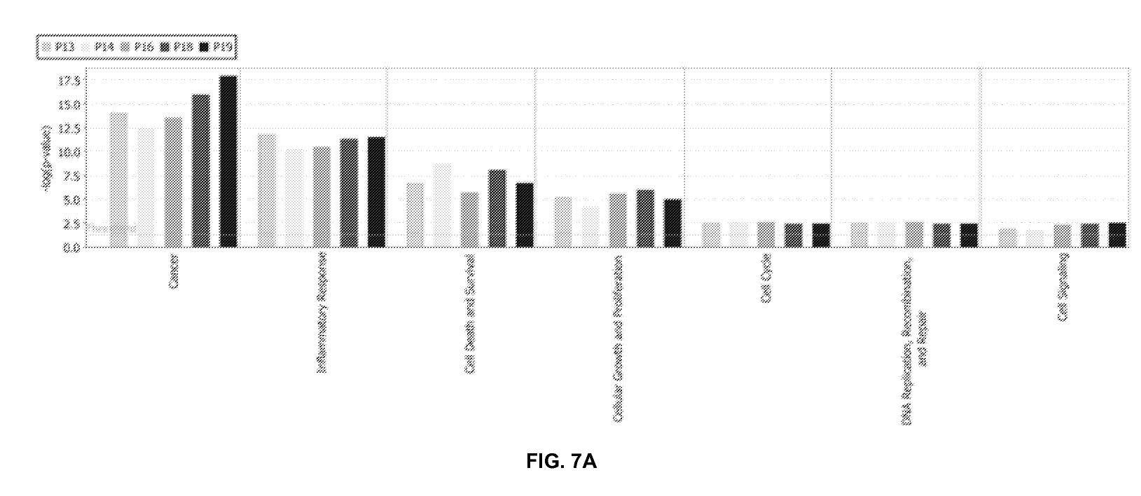

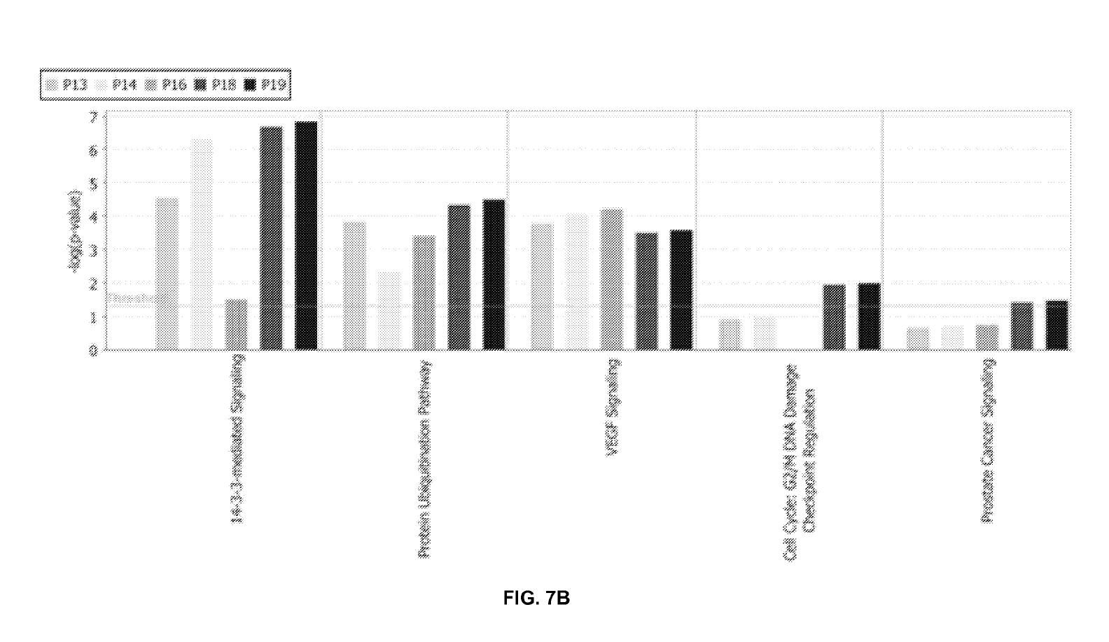

The group of 71 proteins was also analyzed using IPA to determine whether the interaction of these proteins is similar across all of the patients. IPA showed that there is similarity in the level of significance of functions and canonical pathways related to apoptosis and cell survival across all the patients. The data sets from the five patients were compared using a Comparison Analysis Tool in the IPA software. The p-value, in this case, is the measure of the likelihood that the association between a set of genes in the dataset and a related function or pathway is due to random association. The cutoff value for the bar graph is set at p<0.05 (or −log<1.3) as indicated by the threshold line in FIGS. 7A and 7B. All the functions across all the patients show a statistically significant non-random association, and the majority of the pathways across all the patients also show a statistically significant non-random association. The fact that some of the canonical pathways are statistically significant in some patients, but not in others can be attributed to some level of heterogeneity across patients. Nonetheless, the majority of the functions and canonical pathways, the level of homogeneity in protein content, and function and pathway significance can clearly be seen and can definitely prove to be a useful tool in the future development of targeted cancer therapeutics.

### Example 3

### Transfer and Reversal of the Cancer Phenotype in Breast Cancer

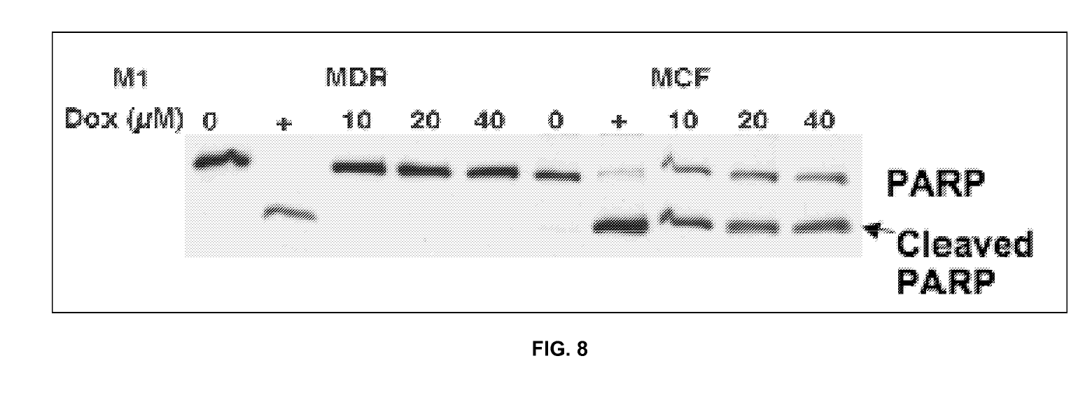

The transfer of chemosensitivity of MCF7 (sensitive) and MCF/MDR (resistant) (MDR) breast cancer cells that are sensitive or resistant, respectively to doxorubicin (Dox) was explored. These cells, similar to the prostate cancer cells, were grown in medium with serum that was depleted of ECVs. The results show that MCF7 cells were sensitive to dose-dependent induction of apoptosis after Dox treatment, but MDR cells remained resistant, as measured by the cleavage of PARP (FIG. 8).

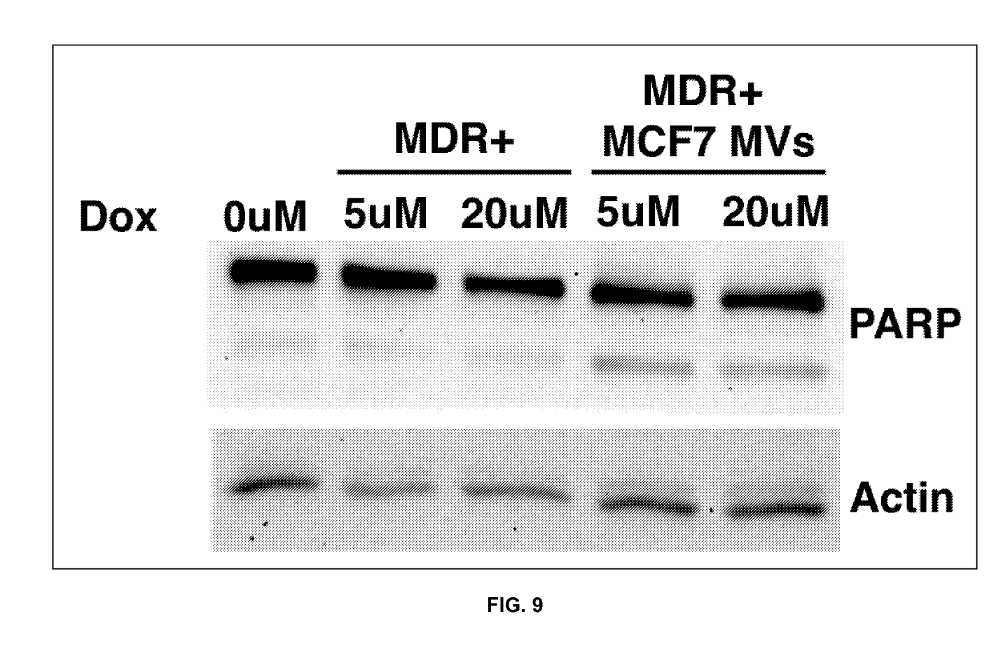

A reciprocal experiment was performed and ECVs from MCF-7 (sensitive) cells were isolated and co-cultured with MDR (resistant) cells for three days. The MDR (resistant) cells were treated with two doses of Dox. As shown in FIG. 9, MDR cells were resistant to the apoptotic effects of Dox as measured by PARP cleavage. However, after the MDR (resistant) cells were co-cultured with the MCF7 (sensitive) ECVs, they underwent apoptosis. Importantly, these results indicate that chemosensitivity can be transferred via ECVs.

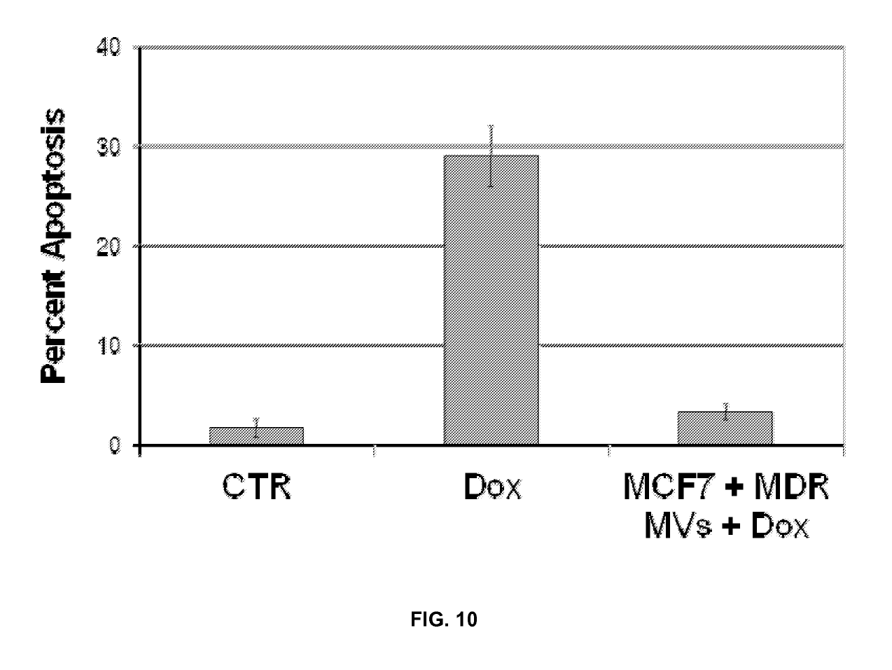

The reciprocal experiment from FIG. 9 was performed, and ECVs were isolated from MDR (resistant) cells and co-cultured with MCF7 (sensitive) cells for three days and treated with 10 nM Dox. The cells were analyzed for apoptosis induction by flow cytometry. As seen in FIG. 10, MCF-7 (sensitive) cells underwent significant apoptosis when treated with 10 nM Dox. However, when co-cultured with MDR (resistant) ECVs, there was a significant reduction in apoptosis (p<0.0005) compared to Dox-treated cells. These results show that chemo-resistance can be transferred via ECVs.

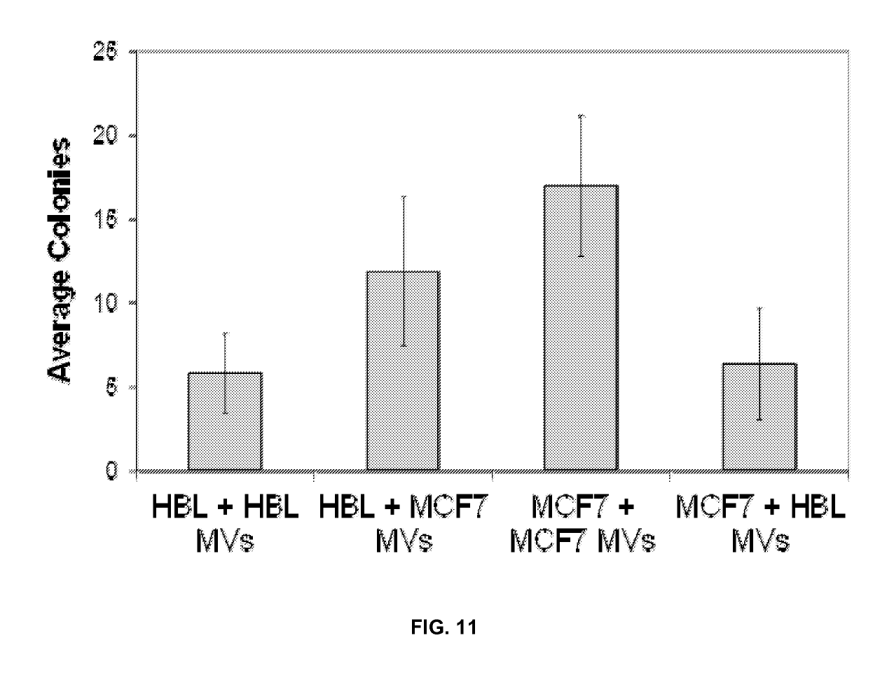

The epithelial HBL-100 (HBL) cell line represents a useful model for studying the progression of human epithelial cells toward malignancy (Caron de Fromentel et al., Exp Cell Res 160:83-94, 1985). A reciprocal transfer of the cancer and non-malignant phenotype between HBL (non-malignant) and MCF7 (malignant) cells via ECVs from these two cell lines was examined. ECVs were isolated from both cell lines and co-culture experiments performed. After five days in culture, cells were harvested and assessed for anchorage-independent growth (soft agar). As shown in FIG. 11, there was a significant increase in anchorage independent growth (p<0.000025) when HBL (non-malignant) cells were incubated with MCF7 (malignant) cell ECVs. This indicates that the cancer phenotype can be transferred via ECVs. In contrast, there was a greater statistically-significant reduction in anchorage-independent growth (p<0.00000012) when MCF7 (malignant) cells were co-cultured with HBL (non-malignant) ECVs. This result demonstrates that cancer can be suppressed via ECVs.

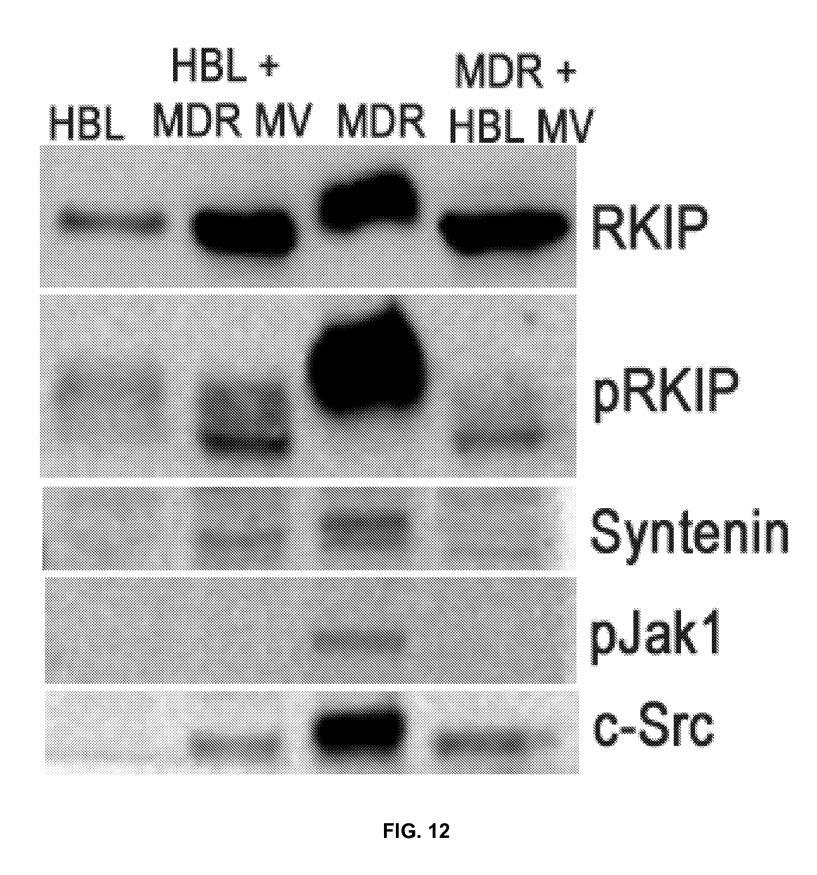

These results demonstrate that the malignant or normal phenotype can be transferred via ECVs. Using stable isotope labeling by amino acids in cell culture (SILAC), a technique based on mass spectrometry that detects differences in protein abundance, the transfer of specific proteins via ECVs to MDR or HBL breast cells was investigated. From the SILAC labeling analysis, a panel of proteins that were transferred to HBL cells via MDR ECVs and to MDR cells via HBL ECVs was discovered. Proteins associated with cell survival and apoptosis signaling based on the results from the SILAC experiment was examined. As shown in FIG. 12, proteins that were selectively transferred or inhibited via HBL or MDR ECVs were detected. Significantly, the transfer of RKIP from MDR ECVS to HBL cells was observed, an effect that may explain the chemosensitivity after transfer. In addition, syntenin, which is a regulator of RKIP was also transferred via ECVs to HBL cells. Phosphorylated Jakl (pJak1) is a pro-survival protein that is not expressed in normal HBL cells (FIG. 12). HBL ECVs completely inhibited pJak1 expression in MDR cells. In contrast the expression of c-Src, another pro-cell survival protein was inhibited by HBL ECVs in MDR cells. These data demonstrate the transfer of specific proteins via ECVs, which may directly affect the phenotype of malignant or normal breast cells.

### Example 4

### Protocol for the Treatment of Cancer

The following example illustrates protocols for use in treating a subject having or at risk for developing cancer. The example also illustrates protocols for treating patients before, during, and/or after surgical procedures, e.g., a surgical procedure to remove a tumor. Skilled practitioners will appreciate that any protocol described herein can be adapted based on a subject's individual needs, and can be adapted to be used in conjunction with any other treatment for cancer.

Amounts of ECVs effective to treat cancer are administered to (or prescribed for) a subject, e.g., by a physician or veterinarian, on the day the subject is diagnosed as having or suffering from cancer, or at risk for developing cancer, e.g., any risk factor associated with an increased likelihood that the subject will develop cancer, e.g., the subject has recently been, is being, or will be exposed to a carcinogen(s)). Subjects are administered, e.g., by exposure, injection, infusion, a therapeutically effective amount of non-cancerous cell ECVs.

If the subject needs to be treated with chemotherapy, radiation therapy, immunotherapy, gene therapy, and/or surgery (e.g., because prescribed by a physician or veterinarian), the subject is treated with ECVs before, during, and/or after administration of the chemotherapy, radiation therapy, and/or surgery. For example, with regard to chemotherapy, immunotherapy, gene therapy, and radiation therapy, ECVs are administered to the subject, intermittently or continuously, starting 0 to 20 days before the chemotherapy, immunotherapy, gene therapy, or radiation therapy is administered (and where multiple doses are given, before each individual dose), e.g., starting at least about 30 minutes, e.g., about 1, 2, 3, 5, 7, or 10 hours, or about 1, 2, 4, 6, 8, 10, 12, 14, 18, or 20 days, or greater than 20 days, before the administration. Alternatively or in addition, ECVs are administered to the patient concurrent with administration of chemotherapy, immunotherapy, gene therapy, or radiation therapy. Alternatively or in addition, ECVs are administered to the patient after administration of chemotherapy, immunotherapy, gene therapy, or radiation therapy, e.g., starting immediately after administration, and continuing intermittently or continuously for about 1, 2, 3, 5, 7, or 10 hours, or about 1, 2, 5, 8, 10, 20, 30, 50, or 60 days, one year, indefinitely, or until a physician determines that administration of the ECVs is no longer necessary.

With regard to surgical procedures, ECVs are administered systemically or locally to a patient prior to, during, and/or after a surgical procedure is performed. Tumorous tissue is directly or indirectly injected, infused, bathed, or otherwise exposed to a therapeutically effective amount of non-cancerous cell ECVs. ECVs are administered to the subject intermittently or continuously, for 1 hour, 2, hours, 3 hours, 4 hours, 6, hours, 12 hours, or about 1, 2, 4, 6, 8, 10, 12, 14, 18, or 20 days, or greater than 20 days, before the procedure. ECVs are administered in the time period immediately prior to the surgery and optionally continue through the procedure, or the administration can cease at least 15 minutes before the surgery begins (e.g., at least 30 minutes, 1 hour, 2 hours 3 hours, 6 hours, or 24 hours before the surgery begins. Alternatively or in addition, ECVs are administered to the patient during the procedure, e.g., by exposure, injection, infusion. Alternatively or in addition, ECVs are administered to the patient after the procedure, e.g., starting immediately after completion of the procedure, and continuing for about 1, 2, 3, 5, 7, or 10 hours, or about 1, 2, 5, 8, 10, 20, 30, 50, or 60 days, 1 year, indefinitely, or until the patient no longer suffers from, or is at risk for, cancer after the completion of the procedure.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating cancer, the method comprising:
selecting a subject having or at risk for developing cancer; and
administering to the subject a therapeutically effective amount of non-cancerous cell extracellular vesicles, to thereby treat cancer in the subject.

2. The method of claim 1, wherein the cancer is selected from the group consisting of prostate cancer, breast cancer, lung cancer, colon cancer, kidney cancer, liver cancer, and brain cancer.

3. The method of claim 1, wherein the non-cancerous cell extracellular vesicles are from the same subject to be treated.

4. The method of claim 1, wherein the non-cancerous cell extracellular vesicles are from non-cancerous cells of the same type as the cancerous cells to be treated in the subject.

5. The method of claim 1, wherein the non-cancerous cell extracellular vesicles are extracellular vesicles of non-cancerous prostate, breast, lung, colon, kidney, liver, or brain cells.

6. The method of claim 1, wherein the cancer is prostate cancer and the non-cancerous cell extracellular vesicles are prostate cell extracellular vesicles.

7. The method of claim 1, wherein the cancer is breast cancer and the non-cancerous cell extracellular vesicles are breast cell extracellular vesicles.

8. The method of claim 1, wherein the cancer is lung cancer and the non-cancerous cell extracellular vesicles are lung cell extracellular vesicles.

9. The method of claim 1, wherein the cancer is colon cancer and the non-cancerous cell extracellular vesicles are colon cell extracellular vesicles.

10. The method of claim 1, wherein the cancer is kidney cancer and the non-cancerous cell extracellular vesicles are kidney cell extracellular vesicles.

11. The method of claim 1, wherein the cancer is liver cancer and the non-cancerous cell extracellular vesicles are liver cell extracellular vesicles.

12. The method of claim 1, wherein the cancer is brain cancer and the non-cancerous cell extracellular vesicles are brain cell extracellular vesicles.

13-18. (canceled)

19. The method of claim 1, wherein the method comprises isolating non-cancerous cell extracellular vesicles from the subject.

20. A pharmaceutical composition comprising:
a substantially pure preparation of non-cancerous cell extracellular vesicles; and
a pharmaceutically acceptable carrier.

21. The composition of claim 20, wherein the composition comprises nanoparticles.

22. The composition of claim 20, wherein the non-cancerous cell extracellular vesicles are extracellular vesicles of non-cancerous prostate cells.

23. The composition of claim 20, wherein the non-cancerous cell extracellular vesicles are extracellular vesicles of non-cancerous breast cells.

24. The composition of claim 20, wherein the non-cancerous cell extracellular vesicles are extracellular vesicles of non-cancerous lung cells.

25. The composition of claim 20, wherein the non-cancerous cell extracellular vesicles are extracellular vesicles of non-cancerous colon cells.

26. The composition of claim 20, wherein the non-cancerous cell extracellular vesicles are extracellular vesicles of non-cancerous kidney cells.

27. The composition of claim 20, wherein the non-cancerous cell extracellular vesicles are extracellular vesicles of non-cancerous liver cells.

28. The composition of claim 20, wherein the non-cancerous cell extracellular vesicles are extracellular vesicles of non-cancerous brain cells.
